SEQUENCE LISTING (1 sequence)

SEQ ID NO: 1 - protein, 52 aa, synthetic construct
  REGION 1..52: note = Synthetic Construct
GGGGSGHEEPCGPSHKSFCLNGGLCYQIPTIPSPFCRCVENYTGARCEKVFL

(19) United States
(12) Patent Application Publication
(10) Pub. No.: US 2025/0339493 A1
(43) Pub. Date: Nov. 6, 2025

(54) METHODS OF USING NEUREGULIN-4 COMPOUNDS

(71) Applicant: ELI LILLY AND COMPANY, Indianapolis, IN (US)

(72) Inventors: Xiaosu MA, Mercer Island, WA (US); Masahiro MURAKAMI, Carmel, IN (US); Wei NI, Indianapolis, IN (US); Jason Dennis ROBARGE, Indianapolis, IN (US)

(21) Appl. No.: 18/869,049

(22) PCT Filed: May 24, 2023
(86) PCT No.: PCT/US2023/067380

§ 371 Date: Nov. 25, 2024

Related U.S. Application Data
(60) Provisional application No. 63/345,658, filed on May 25, 2022.

(51) Int. Cl. A61K 38/18

(52) U.S. Cl. CPC A61K 38/1883

(57) ABSTRACT

The present invention relates to doses and dosing regimens of an NRG4 compound in the treatment of cardiovascular disease (CVD) related conditions, including heart failure (HF).

The present invention relates to the field of medicine. More particularly, the present invention relates to methods of using a Neuregulin-4 compound in the treatment of cardiovascular disease (CVD) and related conditions, including in particular heart failure (HF).

There is an unmet medical need for new and improved treatments for HF. HF is a serious condition that has been estimated at affecting over 6 million adults in the US and at least 26 million people worldwide. The prevalence of HF is predicted to continue to increase significantly. Currently available therapies are intended to slow down disease progression and improve symptoms, and rely on hemodynamic changes to reduce the workload of the failing heart. These therapies include agents intended to: (a) reduce heart rate, such as beta blockers and ivabradine; (b) reduce blood pressure, such as angiotensin-converting enzyme (ACE) inhibitors, angiotensin II receptor blockers (ARB), mineralocorticoid receptor antagonists (MRA), and sacubitril/valsartan (ENTRESTO®); and/or (c) treat or prevent volume overload, such as diuretics and MRA and sodium-glucose co-transporter 2 (SGLT2) inhibitors. These treatments, however, do not directly treat the heart, and have practical limitations, such as requiring dose titration and monitoring for hypotension. Despite improvements in therapies for chronic HF, morbidity and mortality rates remain high. Given the high unmet medical need, new and better treatments for chronic HF are needed.

WO2020/205840 describes NRG4 compounds with utility in the treatment or prevention of CVD and related conditions, including in particular HF with reduced ejection fraction (HFrEF), through mechanisms that potentially involve cardiac tissue repair. A preferred NRG4 compound described comprises the following amino acid sequence:

(SEQ ID NO: 1)
GGGGSGHEEPCGPSHKSFCLNGGLCY
QIPTIPSPFCRCVENYTGARCEKVEL

Specific doses and dosing regimens for treatment with such a compound are needed.

Accordingly, the present invention provides a method of treating HFrEF in a patient in need thereof, comprising administering to the patient an NRG4 compound of SEQ ID NO:1 in a dose between 21-560 mg.

In another aspect, the present invention provides a pump device comprising the NRG4 compound of SEQ ID NO:1 in a dose between 21-560 mg.

In another aspect, the present invention provides an NRG4 compound of SEQ ID NO:1 for use in the treatment of HFrEF in a patient at a dose between 21-560 mg.

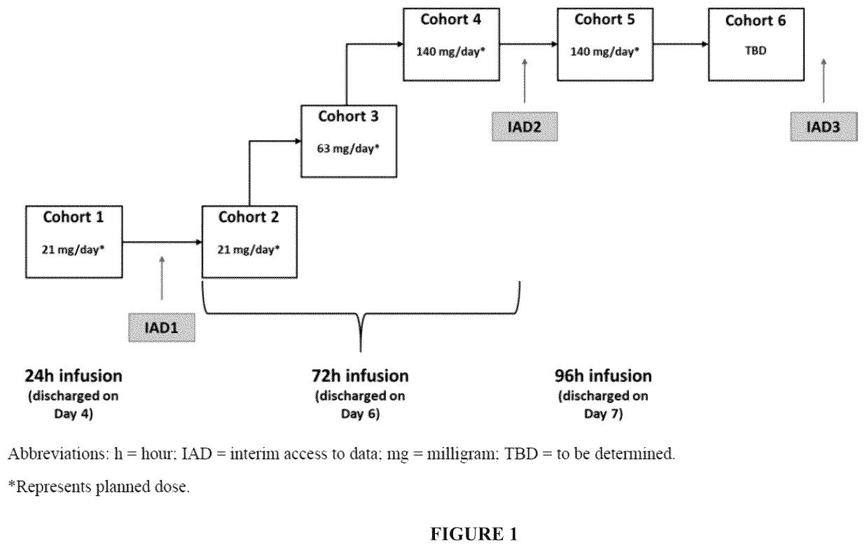

FIG. 1: Scheme for clinical study in patients with HFrEF.

In certain embodiments, the present invention provides a method of treating HFrEF wherein the NRG4 compound of SEQ ID NO:1 is administered in a dose between 21-560 mg. In an embodiment, the dose is between 21-63 mg. In another embodiment, the dose is between 21-140 mg. In another embodiment, the dose is between 21-189 mg. In another embodiment, the dose is between 21-280 mg. In another embodiment, the dose is between 21-420 mg. In another embodiment, the dose is between 21-560 mg. In another embodiment, the dose is between 63-140 mg. In another embodiment, the dose is between 63-189 mg. In another embodiment, the dose is between 63-280 mg. In another embodiment, the dose is between 63-420 mg. In another embodiment, the dose is between 63-560 mg. In another embodiment, the dose is between 140-189 mg. In another embodiment, the dose is between 140-280 mg. In another embodiment, the dose is between 140-420 mg. In another embodiment, the dose is between 140-560 mg. In another embodiment, the dose is between 189-280 mg. In another embodiment, the dose is between 189-420 mg. In another embodiment, the dose is between 189-560 mg. In another embodiment, the dose is between 280-420 mg. In another embodiment, the dose is between 280-560 mg. In another embodiment, the dose is between 420-560 mg.

In certain embodiments, the present invention provides a method of treating HFrEF wherein the NRG4 compound of SEQ ID NO:1 is administered in a dose of about 21 mg, about 63 mg, about 140 mg, about 189 mg, about 280 mg, about 420 mg and about 560 mg. In an embodiment, the dose is about 21 mg. In another embodiment, the dose is about 63 mg. In another embodiment, the dose is about 140 mg. In another embodiment, the dose is about 189 mg. In another embodiment, the dose is about 280 mg. In another embodiment, the dose is about 420 mg. In another embodiment, the dose is about 560 mg.

In certain embodiments, the present invention provides a method of treating HFrEF wherein the NRG4 compound of SEQ ID NO:1 is administered in a dose between 1-60 mg. In certain embodiments, the dose is between 3-30 mg. In certain embodiments, the dose is selected from the group consisting of about 1 mg, about 3 mg, about 10 mg, about 30 mg and about 60 mg.

In certain embodiments, the dose is administered by subcutaneous (SC) infusion over the course of 24-96 hours. In certain embodiments, the dose is administered by subcutaneous (SC) infusion over the course of 24-48 hours. In certain embodiments, the dose is administered by subcutaneous (SC) infusion over the course of 24-72 hours. In certain embodiments, the dose is administered by subcutaneous (SC) infusion over the course of 48-72 hours. In certain embodiments, the dose is administered by subcutaneous (SC) infusion over the course of 48-96 hours. In certain embodiments, the dose is administered by subcutaneous (SC) infusion over the course of 72-96 hours.

In certain embodiments, the dose is administered by SC infusion over the course of about 24 hours. In other embodiments, the dose is administered by SC infusion over the course of about 48 hours. In other embodiments, the dose is administered by SC infusion over the course of about 72 hours. In other embodiments, the dose is administered by SC infusion over the course of about 96 hours.

In an embodiment, the dose is 21 mg administered over the course of about 24, about 48, about 72 or about 96 hours. In an embodiment, the dose is about 63 mg administered over the course of about 24, about 48, about 72 or about 96 hours. In an embodiment, the dose is about 140 mg administered over the course of about 24, about 48, about 72 or about 96 hours. In an embodiment, the dose is about 189 mg administered over the course of about 24, about 48, about 72 or about 96 hours. In an embodiment, the dose is about 280 mg administered over the course of about 24, about 48, about 72 or about 96 hours. In an embodiment, the dose is about 420 mg administered over the course of about 24, about 48, about 72 or about 96 hours. In an embodiment, the dose is 560 mg administered over the course of about 24, about 48, about 72 or about 96 hours.

In certain embodiments, the patient is treated with the dose of NRG4 compound about once a month. In certain embodiments, the patient is treated with the dose of NRG4 compound about once every 2 months. In certain embodiments, the patient is treated with the dose of NRG4 compound about once every 3 months. In certain embodiments, the patient is treated with the dose of NRG4 compound about once every 4 months. In certain embodiments, the patient is treated with the dose of NRG4 compound about once every 5 months. In other embodiments, the patient is treated with the dose of NRG4 compound once about every 6 months.

As used herein, the term “about” is intended to refer to an acceptable degree of error for the amount or quantity indicated given the nature or precision of the measurements. For example, the degree of error can be indicated by the number of significant figures provided for the measurement, as is understood in the art, and includes but is not limited to a variation of +/−1 in the most precise significant figure reported for the amount or quantity. Typical exemplary degrees of error are within 20 percent (%), preferably within 10%, and more preferably within 5% of a given value or range of values. Numerical quantities given herein are approximate unless stated otherwise, meaning that the term “about” can be inferred when not expressly stated.

As used herein, the term “treating” or “to treat” includes restraining, slowing, stopping, or reversing the progression or severity of an existing symptom or disorder. Treatment of heart failure or HFrEF according to the present invention may be reflected in one or more of a variety of measures relevant to heart failure, including, for example improvements in one or more echocardiogram parameters. Examples of such parameters include left atrial volume (LAV); left atrial volume index (LAVI); left ventricular end-diastolic volume (LVEDV); left ventricular end-diastolic volume index (LVEDVI); left ventricular ejection fraction (LVEF); left ventricular end-systolic volume (LVESV); left ventricular end-systolic volume index (LVESVI); left ventricular global circumferential strain (LVGCS); left ventricular global longitudinal strain (LVGLS); left ventricular mass (LVM); left ventricular mass index (LVMI); E wave to A wave ratio (E/A); ratio between early mitral inflow velocity and mitral annular early diastolic velocity (E/e′); left ventricular outflow tract velocity time integral (LVOT-VTI); tricuspid regurgitation peak velocity (TRV); and TR velocity gradient, Estimated Right Ventricle Systolic Pressure. In certain embodiments, the method results in a significant increase in LVEF. In certain embodiments, the method results in a significant reduction in risk of CV death. In certain embodiments, the method results in a significant reduction in risk of HF-related in hospitalization. In certain embodiments, the method results in an improvement in the patient's NYHA class evaluation. In certain embodiments, the method results in an improvement in the patient's functional and symptomatic assessments.

Certain benefits of treatment according to embodiments of the present invention may be achieved after treatment for at least about 1 month. Certain benefits of treatment according to embodiments of the present invention may be achieved after treatment for at least about 3 months. Certain benefits of treatment according to embodiments of the present invention may be achieved after treatment for at least about 6 months. Certain benefits of treatment according to embodiments of the present invention may be achieved after treatment for at least about 1 year.

In certain embodiments, administration of the NRG4 compound of SEQ ID NO:1 according to the present invention results in an improvement in LVEF. In certain embodiments, administration of the NRG4 compound of SEQ ID NO:1 according to the present invention results in at least about a 3% improvement in LVEF In certain embodiments, administration of the NRG4 compound of SEQ ID NO:1 according to the present invention results in at least about a 5% improvement in LVEF. In certain embodiments, administration of the NRG4 compound of SEQ ID NO:1 according to the present invention results in at least about a 5% improvement in L VEF after 6 months of treatment. In certain embodiments, administration of the NRG4 compound of SEQ ID NO:1 according to the present invention results in at least about a 5% improvement in LVEF after 1 year of treatment.

In certain embodiments, administration of the NRG4 compound of SEQ ID NO:1 according to the present invention results in at least about a 5% improvement in LVESV after 1 year of treatment.

In certain embodiments, administration of the NRG4 compound of SEQ ID NO:1 according to the present invention results in significant reductions in LVEDV after 1 year of treatment. In certain embodiments, administration of the NRG4 compound of SEQ ID NO:1 according to the present invention results in a reduction in LVGLS. In certain embodiments, administration of the NRG4 compound of SEQ ID NO: I according to the present invention results in at least about a 1% reduction in LVGLS. In certain embodiments, administration of the NRG4 compound of SEQ ID NO:1 according to the present invention results in at least about a 3.5% reduction in LVGLS. In certain embodiments, administration of the NRG4 compound of SEQ ID NO:1 of the present invention results in at least about a 15% reduction in risk of CV death and/or HF hospitalization.

In certain embodiments, the method results in reductions in the risk of total mortality, reductions in the risk of myocardial infarction (MI), reductions in the risk of stroke, reductions in the risk of need for left ventricular assist device (LVAD) implantation and/or cardiac transplant, improvement in symptoms and physical limitations of heart failure and/or improvement in quality of life (QoL).

In addition, as noted above, administration of the NRG4 compound of SEQ ID NO:1 according to certain embodiments of the presentation is capable of providing improvements in heart failure-related measures, such as those described above, without increasing safety risks. Thus, in preferred embodiments, administration of the NRG4 compound of SEQ ID NO:1 according to the present invention results in no increases in safety risks such as increased hypotension; worsened renal function; electrolyte imbalances; liver dysfunction; or incidence of tumors.

Administration of the NRG4 compound of SEQ ID NO:1 according to the present invention is typically parenteral, e.g., intravenous (IV), subcutaneous (SC) or intraperitoneal (IP). Thus, in certain embodiments of the present invention, the NRG4 compound of SEQ ID NO:1 is administered intravenously. In other embodiments of the present invention, the NRG4 compound of SEQ ID NO:1 is administered intraperitoneally. In other embodiments, the NRG4 compound of SEQ ID NO:1 is administered subcutaneously.

In order to control duration of exposure for optimized and sustained efficacy with sufficient margin to cardiac toxicity, parenteral administration of NRG4 compounds is preferably by continuous or intermittent infusion. For example, many types of infusion pumps are used to infuse a broad range of medications and such pumps may be used in administering NRG4 compounds according to the present invention. More recently, patch pumps have been developed which are smaller than traditional devices and which attach directly to the patient's skin, resulting in minimal interference with patients' day-to-day activities. Thus, in certain embodiments, the NRG4 compound of SEQ ID NO:1 is administered by infusion pump or patch pump. Preferably, the NRG4 compound of SEQ ID NO:1 is administered by patch pump.

As noted above, all HF patients, even those who are mildly symptomatic are at high risk of dying. Thus, when used herein, references to a “patient in need” of a treatment for heart failure (HF) may refer to a broad range of individuals having HF, including those with a broad range disease severity as described below. The New York Heart Association (NYHA) has provided a classification scheme for the degree or severity of HF, as summarized below in Table 1:

TABLE 1

| NYHA | |
|---|---|
| Class | Symptoms |
| I | No limitation of physical activity. Ordinary physical activity does not cause |
| | undue fatigue, palpitation, dyspnea (shortness of breath). |
| II | Slight limitation of physical activity. Comfortable at rest. Ordinary physical |
| | activity results in fatigue, palpitation, dyspnea (shortness of breath). |
| III | Marked limitation of physical activity. Comfortable at rest. Less than |
| | ordinary activity causes fatigue, palpitation, or dyspnea. |
| IV | Unable to carry on any physical activity without discomfort. Symptoms of |
| | heart failure at rest. If any physical activity is undertaken, discomfort |
| | increases. |

In certain embodiments, the patient in need is in heart failure NYHA Class II-IV. In certain embodiments, the patient in need is in heart failure NYHA Class II. In certain embodiments, the patient in need is in heart failure NYHA Class III. In certain embodiments, the patient in need is in heart failure NYHA Class IV. In certain embodiments, the patient in need is in heart failure NYHA Class II-III.

As noted above, existing therapeutic treatment options for heart failure, including current standard of care, improve symptoms and slow down disease progression through hemodynamic mechanisms—e.g., reducing blood pressure, heart rate and/or plasma volume—to reduce the workload of the failing heart. The NRG4 compound of SEQ ID NO:1, by contrast, achieve their effects through a different mechanism of action, namely, selective HER4 binding and the activity resulting therefrom, which directly improves cardiomyocyte survival and cell metabolism and promotes myocardial regeneration. Due to these different mechanisms of action, the NRG4 compound of SEQ ID NO:1 can be administered on top of existing SoC without titration or monitoring. Thus, in certain embodiments, the NRG4 compound of SEQ ID NO:1 may be administered in combination with one or more additional treatments for heart failure. In certain embodiments, the one or more additional treatments for heart failure are selected from the group consisting of beta blockers, ACE inhibitors, ARBs, MRAs, diuretics, ivabradine and sacubitril/valsartan (ENTRESTO®). In certain embodiments, the NRG4 compound of SEQ ID NO:1 may be administered in combination with SGLT2 inhibitors or sGC activators.

The NRG4 compound of SEQ ID NO:1 may also have utility in treatment of other diseases or conditions, including but not limited to, heart failure with preserved ejection fraction (HFpEF), and/or other heart related disorders or conditions.

The invention is further illustrated by the following examples, which are not to be construed as limiting.

### EXAMPLES

### Clinical Study in Healthy Volunteers

A multiple-center, investigator- and participant-blind, randomized, placebo-controlled, single-ascending dose study is designed to evaluate the safety and tolerability of a single subcutaneous (SC) dose or an intravenous (IV) dose of the NRG4 compound of SEQ ID NO:1 in healthy participants. Cohorts 1-5 are intended to receive a single SC dose of the NRG4 compound or placebo. Cohort 6 is used to assess a single IV injection of the NRG4 compound. This cohort is to enable an estimation of absolute bioavailability of the NRG4 compound. Doses for the cohorts are as follows:
Cohort 1:1 mg (6 participants NRG4 compound; 2 participants placebo)·
Cohort 2:3 mg (6 participants NRG4 compound; 2 participants placebo).
Cohort 3:10 mg (6 participants NRG4 compound; 2 participants placebo).
Cohort 4:30 mg (6 participants NRG4 compound; 2 participants placebo).
Cohort 5:60 mg (6 participants NRG4 compound; 2 participants placebo).
Cohort 6 (8 participants 3 mg NRG4 compound IV)

48 participants received a dose of NRG4 compound or placebo, with eight males, and 40 females. The age range of the participants was 25 to 65 years. A stable dose of antihypertensive medication, with the exception of beta blockers, and lipid lowering medication were allowed.

The study intervention was administered either via a single SC injection into the lower abdominal region or as an IV bolus from a syringe to an indwelling venous catheter. Study interventions administered are summarized in Table 2:

TABLE 2

| Treatment | NRG4 compound | Placebo |
|---|---|---|
| Dosage Formulation | 20 mg of lyophilized | Bacteriostatic 0.9% sodium |
| | powder in a vial^{a} | chloride solution in a vial |
| Unit Dose | 10 mg/mL | NA |
| Strength/Dosage Level | | |
| Route of Administration | SC or IV bolus injection | SC or IV bolus injection |
| Dosing Instructions | Single dose | Single dosec |
| | (at the assigned dose level) | (volume matched with |
| | | active drug) |

Abbreviations: IV = intravenous; NA = not applicable; SC = subcutaneous.
^{a}Reconstituted with sterile water for injection.

On Day 1, participants receive a single dose of their assigned treatment (either NRG4 compound or placebo) for first 5 cohorts and NRG4 compound for the final IV cohort. PK sampling and safety assessments, including AEs, medical assessments, clinical laboratory tests, vital signs, and ECGs, were performed at predefined times through discharge from the CRU on Day 4. Dose escalated cohorts were only initiated after satisfactory review of the safety results from all participants receiving NRG4 compound in the preceding cohort through Day 4. Participants returned to the CRU for outpatient visits on Days 7 and 15 for PK, safety, and immunogenicity assessments, as appropriate. Follow-up visit for PK, safety, and immunogenicity monitoring occurred on Day 29.

At the visits and times specified in the protocol venous blood samples of approximately 3 mL each were collected to determine the plasma concentrations of NRG4 compound. PK parameter estimates for NRG4 compound were calculated by standard noncompartmental methods of analysis using Phoenix WinNonlin Version 8.1. Absolute bioavailability (F) was calculated as the ratio of geometric mean of 3 mg SC AUC to 3 mg IV AUC. The PK parameters were summarized and listed separately for SC and IV-dosed cohorts. The plasma concentrations of the NRG4 compound were plotted and summarized. The dose proportionality for SC doses of NRG4 compound were assessed for the log-transformed Cmax, AUC (0-∞), and AUC (0-tlast) using a power model (where log-dose acted as an explanatory variable) to estimate ratios of dose normalized geometric means and corresponding 90% CIs. The estimated ratio of dose-normalized geometric means of PK parameters between the highest and lowest doses were used to assess dose proportionality.

Following single SC dosing, the plasma concentration profile for NRG4 compound was characterized by a rapid absorption phase with median tmax of 2.0 hours (range: 1.00 to 4.08 hours) across the 1 to 60 mg dose range, with mean half-life ranging between 2.20 and 3.21 hours. The geometric mean values of CL/F, Vz/F, and Vss/F ranged from 4.88 to 6.05 L/h, 19.2 to 24.3 L, and 22.5 to 27.5 L across the 1 to 60 mg SC dose range. Variability estimates for AUC (0-0) and Cmax following SC dosing were generally moderate with geometric mean CV % of 16 to 35% and 13 to 37%, respectively. Following administration of SC bolus dose of 1 to 60 mg, the PK of NRG4 compound increase approximately proportionally, with AUC (0-0) slightly higher than proportional and Cmax slightly less than proportional increase with the increase of dose.

Following administration of 3 mg IV bolus dose of the NRG4 compound, plasma concentration of the NRG4 compound appeared to decline in a multiphasic manner with geometric mean t1/2 of 2.53 hours, similar to the geometric mean for 3 mg SC dose (2.45 hours) indicating that the elimination was not route dependent. The geometric mean values of CL, Vz, and Vss were 6.01 L/h, 22.0 L, and 13.6 L for 3 mg IV dose group. Variability estimates for AUC (0-0) and Cmax following IV dosing were moderate with a geometric mean CV % of 26% and 38%, respectively.

The geometric mean F of the NRG4 compound following SC administration relative to IV administration was 104% based on AUC (0-0) and AUC (0-tlast), suggesting near complete absorption. Higher than 100% calculated F might be due to SC and IV data generated from separate cohorts with different baseline characteristics.

The majority of treatment emergent adverse events (TEAEs) were not clinically significant and no deaths, serious adverse events (SAEs) or discontinuations due to adverse events were reported. Based on the low severity of TEAEs and lack of SAEs, the NRG4 compound is deemed safe and well-tolerated upon both single bolus SC and IV dosing.

The safety and PK results support progression to study in patients with HFrEF.

Clinical Study in Patients with HFrEF

A clinical trial is designed to evaluate different dose levels and subcutaneous infusion durations on safety, pharmacokinetics, and pharmacodynamics of the NRG4 compound of SEQ ID NO:1 in participants with chronic heart failure with reduced ejection fraction HFrEF. The study is a multicenter, randomized, placebo-controlled, participant- and investigator-blind, single-ascending dose study in participants with HFrEF. Participants receive a subcutaneous (SC) infusion of either the NRG4 compound of SEQ ID NO:1 or placebo.

Approximately 50 participants are enrolled to study intervention such that approximately 36 to a maximum of 49 evaluable participants complete the study.

Criteria for participants to be eligible include the following: Are aged 18 to 75 years (20 to 75 years for Japanese participants), inclusive, at the time of signing the informed consent; Have a body mass index <40 kg/m2 at screening; Have venous access sufficient to allow for blood sampling and administration of study intervention as per the protocol; Have chronic stable HF (NYHA classification II and III) on guideline-directed HF therapy for at least 6 months prior to enrollment. Have not changed optimal guideline-directed HF therapy, either medication or medication dose, in the last 1 month prior to screening and during screening period, and do not plan to change HF therapy for the next 3 months; If treated with oral diuretics, dose must be stable for at least 2 weeks prior to screening and remain at a stable dose prior to enrollment; Have LVEF <40% measured quantitatively using ECHO, computed tomography angiogram, or cardiac magnetic resonance imaging in an historical imaging assessment performed within 12 months prior to screening, as well as by ECHO at screening as determined by the central ECHO laboratory; Have a record of NT-proBNP ≥300 μg/mL within the past 6 months prior to screening; Have an estimated glomerular filtration rate ≥30 mL/minute/1.73 m2 at screening.

Criteria for exclusion from the study include the following: Have myocardial infarction, coronary artery bypass graft surgery, revascularization or other major cardiovascular surgery, stroke, or transient ischemic attack in the last 90 days prior to screening; Have acute decompensated HF (exacerbation of HF) requiring IV diuretics, IV inotropes, or IV vasodilators, within 12 weeks prior to screening, and/or during screening period until randomization; Have SBP ≥180 mmHg at screening; Have SBP >160 mmHg both at screening and randomization; Have symptomatic hypotension or SBP <90 mmHg at screening or randomization; Have resting heart rate >90 bpm at screening or randomization; Have unstable angina pectoris or exercise-induced angina within 30 days prior to screening; Have known cardiac amyloidosis, accumulation diseases (e.g. haemochromatosis, Fabry disease), muscular dystrophies, cardiomyopathy with reversible causes (e.g. stress cardiomyopathy), hypertrophic cardiomyopathy, pericardial constriction, or complex congenital heart disease; Have any moderate-to-severe stenosis of the mitral and/or aortic valve or moderate-to-severe or greater mitral and/or aortic regurgitation, as determined by the central ECHO laboratory at the screening ECHO; Have a left bundle branch block at screening, whether complete or incomplete; Have a history or presence of hepatic, pancreatic, or biliary tract disorders that, in the investigator's opinion, may confound safety assessment; Have any of the following, as determined by the local laboratory during screening: ALT or AST level >2X the ULN for the reference range, or ALP level >1.5X the ULN for the reference range, or TBL above the ULN for the reference range; Have a history of malignancy or active malignancy at screening; Have not had age-appropriate cancer screening as recommended by the American Cancer Society (Smith et al. 2019) or local guidelines in Japan (MHLW 2016) and per medical judgment; Have a suboptimal ECHO image quality assessed by central laboratory at screening; Have been listed for cardiac transplantation and/or anticipated/implanted ventricular assist device; Have had cardiac resynchronization therapy implanted within 6 months; Have shown evidence of hepatitis C and/or positive hepatitis C antibody at screening; Have shown evidence of hepatitis B and/or positive hepatitis B surface antigen at screening; Have shown evidence of human immunodeficiency virus infection and/or positive human immunodeficiency virus antibodies at screening; Are using cardiac contractility modulation therapy.

A total of 6 cohorts of participants will each receive a single continuous SC infusion of either NRG4 compound or placebo (4 NRG4 compound: 2 placebo, 5 NRG4 compound: 3 placebo, or 6 NRG4 compound: 3 placebo. The planned dose levels for Cohorts 1 to 5 are 21, 63, and 140 mg/day for 24 to 96 hours (total dose range: 21 to 560 mg) to explore the exposure-response range after SC infusion with different infusion duration in participants with chronic HFrEF.

The dose level and infusion duration for Cohort 6 is determined after a review of safety and available PK and PD data from Cohorts 1 to 4. The total exposure (area under the concentration-time curve [AUC]) of Cohort 6 will not exceed the maximum exposure already tested in the preceding cohorts.

By nature of being a dose-escalation study, data are evaluated on an ongoing basis until the highest planned dose of NRG4 compound is reached. The decision to proceed to the next dose level of NRG4 compound is made by the sponsor and the investigator based on safety and tolerability data obtained from a minimum of 6 participants at the prior dose level during their inpatient stay. The study design is depicted in FIG. 1.

Participants are required to attend the following visits at the investigative site: a screening visit occurring up to 45 days prior to dose administration; admission to site on Day-1 for eligibility confirmation, randomization, and start of inpatient stay; a 4- to 7-day inpatient treatment visit; an outpatient visit on Day 15; 3 follow-up visits on Days 30, 60, and 90.

On Day 1, participants receive a continuous SC infusion of either NRG4 compound or placebo. The length of the inpatient stay depends on the duration of the infusion per cohort. Participants are discharged from the investigative site 48 hours after the end of the treatment period.

Participants are discharged on: Day 4 for a 24-hour infusion (Cohort 1); Day 6 for a 72-hour infusion (Cohorts 2, 3, and 4); and Day 7 for a 96-hour infusion (Cohort 5).

The dose, infusion duration, and length of inpatient stay for Cohort 6 is determined after a review of safety and available PK and PD data from Cohort 1 to Cohort 4 at minimum. The dose level of Cohort 6 will not exceed the maximum exposure already tested in the preceding cohorts.

All safety data are listed and summarized using standard descriptive statistics. Safety parameters that are assessed include safety laboratory parameters, vital signs, and AEs. The parameters are summarized with regard to observed values and change from baseline by treatment group at each time point using descriptive statistics. In addition, all clinical chemistry, hematology, and urinalysis data outside the reference ranges are tabulated by parameter and treatment group. Vital signs are summarized with regard to observed values and change from baseline (Day 1, predose) by treatment at each time point using descriptive statistics. Other parameters that are assessed include cardiac troponin T and creatine kinase.

Pharmacokinetics parameter estimates for NRG4 compound are calculated using standard noncompartmental methods of analysis. The primary parameters for analysis are Cmax, Css, and AUC. Other noncompartmental parameters such as half-life, CL/F, and apparent volume of distribution may be reported.

For pharmacodynamics, the primary parameters for echocardiography analysis include LVEF, left ventricular global longitudinal strain, and other echocardiogram parameters. PD data and AUC of PD data (both actual value and change from baseline) are summarized using descriptive statistics. A longitudinal linear model analyzes change from baseline for LVEF and AUC of LVEF.

Exploratory endpoints also include assessment of changes in participant symptomatic status following administration of either NRG4 compound or placebo, including NYHA class evaluation.

Safety and tolerability assessments are made over all dose levels, including incidence of AEs and evaluation of vital signs, ECGs, and clinical safety laboratory tests. The decision to escalate doses between the cohorts are based primarily on safety and tolerability data from preceding dose cohorts and within the cohort.

The planned dose levels of 21, 63, and 140 mg/day for 24 to 96 hours (total dose range: 21 to 560 mg) are selected with an emphasis on optimizing the benefit-risk for participants while ensuring the evaluation of exposure-response relationships after SC infusion with different infusion duration in participants with chronic HFrEF. To ensure that early cohorts have a potential for therapeutic benefit, the starting dose is predicted to achieve approximately 20% of the ED₂₀ for improved LVEF.

Based on preliminary PK results from a single-ascending dose study, the mean CL/F ranges from 4.9 to 6.05 L/h after a single SC dose of 1 to 30 mg, and t½ is approximately 2.5 to 3 hours in healthy volunteers. Based on the nonclinical PK and efficacy assessments in male Sprague Dawley rats with myocardial infarction, the NRG4 compound exposure for achieving 50% of maximum effect (EAUC₅₀) on ejection fraction is approximately 2700 nM·h (14.8 μg·h/mL).

Table 3 presents the estimated margins of safety of 21 mg/day infused for 24 hours and 140 mg/day infused for 96 hours doses of NRG4 compound. The anticipated therapeutic benefit for patients with HFrEF outweighs any potential risks identified in the nonclinical safety profile.

TABLE 3
Margin of safety for continuous subcutaneous infusion
of NRG4 compound based on predicted exposure.

| | | Duration | | | Cave, ss | |
|---|---|---|---|---|---|---|
| | | of | | | Multiple^{e} | |
| | Dose | Infusion | AUC0-t | Cave, ss^{d} | Start | Max |
| | (mg/kg/day) | (h) | (μg · h/mL) | (μg/mL) | Dose | Dose |
| Human^{a} | | | | | | |
| Planned Start | 0.3 | 24 | 3.96 | 0.17 | — | — |
| dose: 21 mg/day | | | | | | |
| Planned Max | 2 | 96 | 105.6 | 1.1 | — | — |
| dose: 140 mg/day | | | | | | |
| Rat Continuous | | | | | | |
| SC Infusion^{b} | | | | | | |
| LOAEL (M) | 1.6 | 96 | 7.17 | 0.072 | 0.42 | 0.07 |
| Non-repro | 16.1 | 96 | 53.3 | 0.54 | 3.2 | 0.5 |
| NOAEL | | | | | | |
| (F) | | | | | | |
| Highest Dose | 202 | 96 | 1140 | 11.5 | 68 | 10 |
| Tested (M + F) | | | | | | |
| Monkey | | | | | | |
| Continuous | | | | | | |
| SC Infusion^{c} | | | | | | |
| NOAEL (M + F) | 12.6 | 96 | 148 | 1.49 | 8.8 | 1.4 |
| Highest Dose | 156 | 96 | 1840 | 18.6 | 109 | 16.9 |
| Tested | | | | | | |

Abbreviations: AUC0-t = area under the concentration versus time curve from time zero to the end of infusion; Cave, ss = average steady-state plasma concentration; F = female; HDT = highest dose tested; LOAEL = lowest-observed-adverse-effect level; M = male; max = maximum; NOAEL = no-observed-adverse-effect level; non-repro NOAEL = no-observed-adverse-effect level for nonreproductive effects (e.g. increased epithelial cell proliferation); PK = pharmacokinetics; SC = subcutaneous. Note: Calculations are based on a human body weight of 70 kg.
^{a}Plasma PK parameters shown were estimated based on preliminary PK data from healthy participants . Doses shown are the starting and highest proposed clinical doses;
^{b}Rat continuous infusion toxicology Study;
^{c}Cynomolgus continuous infusion toxicology study;
^{d}Cave, ss in animals was calculated by dividing observed AUC0-99 during SC infusion in animals by 99 h; Cave, ss in human was calculated as infusion rate / (CL/F) during SC infusion;
^{e}Cave, ss multiple = mean Cave, ss in animal/predicted mean Cave, ss in human.

The investigative site personnel administers either NRG4 compound or placebo as a continuous SC infusion while participants are inpatient (Table 4). The investigative site personnel dispense doses according to the randomization scheme provided by the sponsor.

TABLE 4
Treatments administered.

| Treatment | NRG4 compound | Placebo |
|---|---|---|
| Dosage Formulation | 20 mg of lyophilized | Bacteriostatic 0.9% sodium |
| | powder in a vial^{a} | chloride solution in a vial |
| Route of Administration | SC infusion | SC infusion |
| Dosing Instructions^{b} | Single continuous dose^{c} | Single continuous dose^{c} |
| | (at the assigned dose level) | (volume matched with |
| | | active drug) |

Abbreviations: mg = milligram; mL = milliliter; NA = not applicable; SC = subcutaneous.
^{a}Reconstitute with bacteriostatic water for injection;
^{b}Flow rate, concentration, total volume to be infused, and other details of dosing instructions will be provided in the Pharmacy Instructions;
^{c}Single dose may require multiple vials of dosage formulation.

The investigative site personnel use a syringe pump to administer continuous SC infusions of the study intervention for the duration stated. SC infusion using a syringe pump, extension tubing, and a needle are used for the administration of study intervention. The SC tissue of the lower abdominal region approximately 5 cm from the umbilicus in the left lower quadrant is used. If the infusion cannot be administered in the left lower quadrant, infusion may be done in the lower right quadrant. For infusions longer than 24 hours, a new SC infusion site and line is to be inserted every 24 hours, according to the Pharmacy Instructions. Change-over of the infusion line every 24 hours occurs as soon as possible and take no longer than 15 minutes. The investigative site personnel use the same type of needle (infusion cannula) for all participants to ensure that infusions are delivered to a consistent target depth into the SC space.

To be eligible, the participants are on background standard of care therapies for HF according to the American College of Cardiology Foundation/American Heart Association (ACCF/AHA) guidelines. Standard evidence-based treatments include either an angiotensin-converting enzyme (ACE) inhibitor, angiotensin receptor blockers (ARBs), or sacubitril/valsartan in combination with a beta-blocker, as well as mineralocorticoid receptor antagonists or SGLT2i where appropriate unless contraindicated or not tolerated. Most participants also require a diuretic agent to control symptoms as well as volume overload.

Participants remain on stable doses of medications which allows assessment of incremental effect of NRG4 compound.

Data are evaluated on an ongoing basis until the highest planned dose is reached. The decision to proceed to the next dose level is made by the sponsor and the investigator based on safety and tolerability data obtained from a minimum of 6 participants at the prior dose level during their inpatient stay. Participants are discharged on Day 4 for a 24-hour infusion, Day 6 for a 72-hour infusion, and Day 7 for a 96-hour infusion. In addition, for the first dose escalation from Cohort 1 to 2, PK results from Cohort 1 (Cmax, Css, and AUC) are used as supporting data for dose escalation. For the remaining dose escalations, PK data are reviewed, if available, but such data are not required. Cohort 6 dose escalation is determined based on the safety, PD, and available PK data, at minimum, from Cohorts 1 to 4.

Table 5 presents the ECHO measurements at each time point.
Echocardiogram Parameters

| | |
|---|---|
| 2D echo | LV volumes |
| | LVEDV (LVEDVI) |
| | LVESV (LVESVI) |
| | LVEF |
| | LAV (LAVI) |
| | LVM (LVMI) |
| Doppler | E/A |
| | TVR |
| | CO (LVOT VTI) |
| Tissue Doppler | E/e′ |
| Color Doppler (Qualitative | Mitral regurgitant severity |
| Measurements only) | Aortic regurgitant severity |
| | Tricuspid regurgitant severity |
| Speckle tracking | LVGLS |
| | LVGCS |

Abbreviations: CO = cardiac output; E/A = E wave to A wave ratio; E/e′ = ratio between early mitral inflow velocity and mitral annular early diastolic velocity; LAV = left atrial volume; LAVI = left atrial volume index; LV = left ventric(le/ular); LVEDV = left ventricular end-diastolic volume; LVEDVI = left ventricular end-diastolic volume index; LVEF = left ventricular ejection fraction; LVESV = left ventricular end-systolic volume; LVESVI = left ventricular end-systolic volume index; LVGCS = left ventricular global circumferential strain; LVGLS = left ventricular global longitudinal strain; LVM = left ventricular mass; LVMI = left ventricular mass index; LVOT = left ventricular outflow tract; VTI = velocity time integral..

At pre-specified visits and times, samples for measurement of NT-proBNP are collected for analysis. NT-proBNP is determined as a PD marker in this study and analyzed at a central laboratory. The NT-proBNP results from predose onwards are blinded to study site.

Biomarker research is performed to address questions of relevance to drug disposition, target engagement, PD, mechanism of action, and variability of participant response, including safety, and clinical outcome. Sample collection is incorporated into clinical studies to enable examination of these questions through measurement of biomolecules including DNA, RNA, proteins, lipids, and other cellular elements.

Serum or plasma samples for non-pharmacogenetic biomarker research are collected at the times specified where local regulations allow. Samples are used for research on the drug target, disease process, variable response to the NRG4 compound, pathways associated with chronic HF, mechanism of action of the NRG4 compound, and/or research method, or for validating diagnostic tools or assay(s) related to chronic HF.

The incidence of AEs for each treatment are presented by severity and by association with investigational product as perceived by the investigator. AEs reported to occur prior to study enrollment are distinguished from those reported as new or increased in severity during the study. The number of investigational product-related SAEs are reported.

All safety data are listed and summarized using standard descriptive statistics. Safety parameters that ware assessed include safety laboratory parameters, vital signs, and AEs. The parameters are summarized with regard to observed values and change from baseline by treatment group at each time point using descriptive statistics. In addition, all clinical chemistry, hematology, and urinalysis data outside the reference ranges are tabulated by parameter and treatment group. Vital signs are summarized with regard to observed values and change from baseline (Day 1, predose) by treatment at each time point using descriptive statistics. Other parameters assessed include cardiac troponin T and creatine kinase.

PK parameter estimates for the NRG4 compound are calculated using standard noncompartmental methods of analysis. The primary parameters for analysis are Cmax, Css, and AUC of the NRG4 compound. Other noncompartmental parameters such as half-life, CL/F, and apparent volume of distribution may be reported. The descriptive statistics for the PK parameters are provided for each dose level or infusion duration. Where appropriate, geometric mean and geometric coefficient of variation are reported. The dose proportionality for the NRG4 compound may be assessed for AUC, Cmax, and Css using a power model to estimate the slope of the power model, which represents the ratio of dose-normalized geometric means, if data allows.

The primary parameters for PD analysis by echocardiography include LVEF, LVGLS, and other parameters listed in Table 5 above. PD data and AUC of PD data (both actual value and change from baseline) are summarized using descriptive statistics.

The actual value change from baseline for LVEF, LVGLS, and other PD parameters, in addition to the AUC of these parameters are summarized for each dose level. A longitudinal linear model analyzes change from baseline for LVEF and AUC of LVEF.

Exploratory graphical PK/PD analyses relating serum exposure of the NRG4 compound to change from baseline in LVEF may be conducted. PK/PD and exposure-response models may be explored to characterize the relationship of the NRG4 compound concentrations and the changes in other biomarkers or outcome measures, provided data are sufficient.

1. A method of treating heart failure with reduced ejection fraction (HFrEF) in a patient in need thereof, comprising administering to the patient an NRG4 compound of SEQ ID NO:1 in a dose between 21-560 mg.

2. The method of claim 1 wherein the dose is selected from the group consisting of about 21 mg, about 63 mg, about 140 mg, about 189 mg, about 280 mg, about 420 mg and about 560 mg.

3. The method of claim 2 wherein the dose is administered by subcutaneous (SC) infusion over the course of 24-96 hours.

4. The method of claim 1 wherein the dose is about 280 mg administered over the course of about 48 hours.

5. The method of claim 1 wherein the dose is about 420 mg administered over the course of about 72 hours.

6. The method of claim 1 wherein the dose is about 560 mg administered over the course of about 96 hours.

7. The method of claim 1 wherein the patient is treated with the dose of NRG4 compound about once every 3 months.

8. The method claim 1 wherein the patient is treated with the dose of NRG4 compound about once every 6 months.

9. The method of claim 1 wherein the NRG4 compound is administered with a pump device.

10. The method of claim 9 wherein the pump is a syringe pump or a patch pump.

11. The method of claim 1, wherein the method results in an improvement in one or more echocardiogram parameters selected from the group consisting of left atrial volume (LAV); left atrial volume index (LAVI); left ventricular end-diastolic volume (LVEDV); left ventricular end-diastolic volume index (LVEDVI); left ventricular ejection fraction (LVEF); left ventricular end-systolic volume (LVESV); left ventricular end-systolic volume index (LVESVI); left ventricular global circumferential strain (LVGCS); left ventricular global longitudinal strain (LVGLS); left ventricular mass (LVM); left ventricular mass index (LVMI); E wave to A wave ratio (E/A); ratio between early mitral inflow velocity and mitral annular early diastolic velocity (E/e′); left ventricular outflow tract-velocity time integral (LVOT-VTI); tricuspid regurgitation peak velocity (TRV); and TR velocity gradient, Estimated Right Ventricle Systolic Pressure.

12. The method of claim 1, wherein the method results in a significant increase in LVEF.

13. The method of claim 1, wherein the method results in a significant reduction in risk of CV death.

14. The method of claim 1, wherein the method results in a significant reduction in risk of HF-related in hospitalization.

15. The method of claim 1, wherein the patient has NYHA II or III heart failure.

16. The method claim 1, wherein the method results in an improvement in the patient's NYHA class evaluation.

17. A pump device comprising an NRG4 compound of SEQ ID NO:1 in a dose between 21-560 mg.

18. The pump device of claim 17, wherein the dose is selected from the group consisting of about 21 mg, about 63 mg, about 140 mg, about 189 mg, about 280 mg, about 420 mg and about 560 mg.

19-29. (canceled)
